(19) United States
(12) Patent Application Publication
(10) Pub. No.: US 2003/0139945 A1
(43) Pub. Date: Jul. 24, 2003

(54) Apparatus and method for constructing and managing clinical rules

(75) Inventors: Kenneth J. Brown, Yorktown Heights, NY (US); William D. Tobin, Franklin Lakes, NJ (US); Glen D. Stettin, Upper Saddle River, NJ (US); Paul R. Brisson, Pelham Manor, NY (US)

Correspondence Address: HALE & DORR LLP, THE WILLARD OFFICE BUILDING, 1455 PENNSYLVANIA AVE, NW, WASHINGTON, DC, 20004, US

(73) Assignee: Medco Health Solutions, Inc., Franklin Lakes, NJ

(21) Appl. No.: 10/337,371

(22) Filed: Jan. 7, 2003

Related U.S. Application Data
(60) Provisional application No. 60/349,349, filed on Jan. 22, 2002.

(51) Int. Cl. G06F 17/60; A61B 5/00

(52) U.S. Cl. 705/2; 600/300; 705/3

(57) ABSTRACT

An apparatus and method are disclosed for defining and managing clinical rules. A computer system provides access to administrators for defining and entering the details of clinical rules for various clients. The administrator selects the prescription products, or medication, for which the clinical rule will apply. The administrator also specifies the coverage criteria to be used for approving coverage of the prescription products. When a prescription claim is submitted to the computer system, the appropriate clinical rule is selected and applied to determine if the cost of the prescription product should be covered.

### RELATED APPLICATION

This application claims the priority of U.S. Provisional Application No. 60/349,349 filed Jan. 22, 2002, which is incorporated herein by reference.

### BACKGROUND OF THE INVENTION

1. Technical Field

The present invention relates to pharmaceutical prescription products and, more particularly to an apparatus and method for creating, modifying, and maintaining clinical rules governing the dispensing of pharmaceutical products in prescription benefit management programs.

2. Description of the Related Art

Employers often provide employees with various benefits upon commencing their employment. These benefits typically include coverage for healthcare and prescription products. The healthcare benefits package is generally provided through a healthcare provider. The specific coverage offered to an employee can depend on several factors, including the particular coverage program negotiated by the employer. For example, the benefits available can be different depending on the medical coverage desired, the prescription medication available, etc. Furthermore, the specific benefits requested will directly effect the coverage cost.

Regardless of the coverage, the healthcare provider will place certain restrictions and/or limitations on the prescription medication (or products) available. These restrictions determine whether the healthcare provider will cover the cost of a prescription claim in full in part, or reject coverage of the prescription claim altogether. For example, the healthcare provider may cover the cost of a prescription claim in full, if the employee is willing to substitute a generic form of the prescribed medication, or try a different (e.g., milder, for example) prescription medication capable of treating the same illness. The cost of the prescription medication can be subsidized to different degrees, depending on the type of coverage, if the employee prefers to use a brand name form of the medication.

Healthcare providers utilize clinical rules to define the type of coverage available to each client, or employer. The clinical rules define the specific parameters for approving a specific prescription therapy, determining whether a prescription will be covered, covering the cost of filling the prescription, etc. Each clinical rule involves a complex decision process which ultimately determines whether a particular medication (or prescription product) will be covered by the healthcare provider. The clinical rule may take into account the patient's age, gender, prior medical conditions, history with a particular type of medication, etc. Accordingly, each clinical rule can easily entail numerous decision-making steps to determine whether the cost of a prescription medication (or product) will be covered, in whole or in part, for a patient. This process must be carried out each time a prescription is filled. Furthermore, the healthcare provider has numerous clients, each of whom requires implementation of numerous clinical rules.

The creation and modification of new clinical rules has traditionally required extensive interaction between the healthcare provider and the pharmaceutical company creating and/or implementing the clinical rules. First, new clinical rules must be designed based on the needs of the healthcare provider. The clinical rules must then be programmed and linked to a clinical rules system. Finally, the clinical rules must be tested by both users and programmers. These tests are often extensive in order to insure that the healthcare provider's requirements have been met.

Maintaining and modifying the clinical rules to accommodate client needs can often prove to be a challenging task requiring months to complete. In addition, such a process is inherently error prone due to the high number of decision making steps that must be created and/or modified. As a clinical rule is being developed or modified, the healthcare provider may still be responsible for providing prescription coverage to the client. Errors in a clinical rule can often result in situations where the health provider must cover the cost a prescription that would normally not be covered. Over time, these costs can quickly accumulate, particularly when a clinical rule requires an extended period of time to create.

Accordingly, there exists a need for a clinical rules system which addresses at least some of the shortcomings of existing systems.

There also exists a need for a clinical rules system capable of reducing the amount of time required to create, modify, and maintain clinical rules.

There exists a further need for a clinical rules system capable of reducing the level of complexity associated with the creation, modification, and maintenance of clinical rules.

There exists a still further need for a clinical rules system capable of reducing the level of interaction required between healthcare providers and clinical rule administrators to create and modify clinical rules.

There also exists a further need for a clinical rules system capable of reducing the number of errors that can occur when implementing the clinical rules.

There also exists a still further need for a clinical rules system capable or reducing the cost of creating, modifying, and maintaining clinical rules.

### SUMMARY OF THE INVENTION

It is therefore one feature and advantage of the present invention to address at least some of the shortcomings of the prior art in regards to clinical rules.

It is an optional feature and advantage of the present invention to provide a clinical rules system capable of reducing the amount of time required to create, modify, and maintain clinical rules.

It is another optional feature and advantage of the present invention to provide a clinical rules system capable of reducing the level of complexity associated with the creation, modification, and maintenance of clinical rules.

It is yet another optional feature and advantage of the present invention to provide a clinical rules system capable of reducing the level of interaction required between healthcare providers and clinical rule administrators to create and modify clinical rules.

It is a further optional feature and advantage of the present invention to provide a clinical rules system capable of reducing the number of errors that can occur when implementing the clinical rules.

It is a still further optional feature and advantage of the present invention to provide a clinical rules system capable or reducing the cost of creating, modifying, and maintaining clinical rules.

The foregoing, and various other needs, are addressed, at least in part, by the present invention, wherein a method and apparatus are provided for efficiently creating, modifying, and maintaining clinical rules for multiple prescription benefit plans by allowing simplified selection of treatments and coverage criteria for prescription products.

According to one embodiment of the invention, a method of creating clinical rules for approving coverage of prescription products comprises the sequential, non-sequential or sequence independent steps: selecting at least one service classification for a clinical rule; selecting at least one prescription product for the clinical rule; selecting at least one treatment type for each selected prescription product; selecting predetermined medical conditions treatable by the prescription products under each clinical rule, based on the defined service classifications; and specifying at least one coverage criteria for approving coverage of each selected prescription product. According to such a method, clinical account executives (CAEs), are capable of quickly and efficiently defining and modifying clinical rules for healthcare providers. Thus, various cost benefits can be achieved as a result of the reduced time. Furthermore, the amount of time required to define clinical rules can be reduced because extensive programming is unnecessary.

According to an optional embodiment of the present invention, various messages can be entered and displayed to various individuals. For example, clinical messages can be specifically tailored to physicians and pharmacists to provide information regarding coverage of a prescription product. Patient messages can be designed to educate a patient on the advantages and disadvantages of a prescription product, and/or on the general requirements for coverage.

According to other optional embodiments of the invention, various physical criteria can be selected to define the clinical rules. For example, a clinical rule can take into account a patient's age, weight, gender, etc. The clinical rules can also have simple and/or compound logic steps to determine whether or not a prescription product will be covered. Certain prescription products can be covered based on the manner in which the medication (or prescription product) will be delivered to the patient. For example, a prescription product may be covered if ingested in the form of a pill, but not covered if taken as a syrup or inhalant. Additionally, various treatment types such as quantity, duration, step therapy, etc., can be used to determine whether or not a prescription product will be covered. Furthermore, the patient's clinical history can be reviewed to determine if prescription products will react with the patient's other prescription products or medical conditions.

According to another aspect of the present invention, a method of evaluating prescription claims using predefined clinical rules, comprises the sequential, non-sequential or sequence independent steps: determining if one or more prescription products in a prescription claim are included in a client's prescription benefits plan; retrieving a plurality of clinical rules from the client's prescription benefit plan; and applying an appropriate clinical rule to each prescription product to determine if the prescription claim should be covered, at least in part, with respect to the prescription products. According to such a method, prescription claims can be quickly and efficiently processed, while minimizing potential errors.

According to an optional embodiment, the present invention can identify the severity of a patient's illness or medical condition. More particularly, the patient's clinical history would be reviewed to identify prescription products which have previously been prescribed to the patient. Next, medical conditions that are treatable by identified prescription products are identified. The medical conditions and prescription products are then compared and analyzed to identify correlations suggesting the severity of the medical conditions.

There has thus been outlined, rather broadly, the more important features of the invention and several, but not all, embodiments in order that the detailed description thereof that follows may be better understood, and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These, together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

FIG. 1 is a block diagram illustrating an arrangement for managing clinical rules according to an exemplary embodiment of the present invention;

FIG. 2 is an illustration of a clinical rule description arrangement according to an exemplary embodiment of the present invention;

FIG. 3 is an illustration of the details for defining clinical rules according to an exemplary embodiment of the present invention;

FIG. 4 is an illustration of the details of conducting searches according to an exemplary embodiment of the present invention;

FIG. 5 is illustrates modification of clinical rules according to an exemplary embodiment of the present invention;

FIG. 6 is an illustration of service classification for clinical rules according to an exemplary embodiment of the present invention;

FIG. 7 is a flow chart illustrating the steps performed in defining clinical rules according to an exemplary embodiment of the present invention;

FIG. 8 is a flow chart detailing construction of clinical rules according to an exemplary embodiment of the present invention;

FIG. 9 is a flow chart detailing selection of treatment types according to an exemplary embodiment of the present invention;

FIG. 10 is a flow chart illustrating application of a clinical rule according to an exemplary embodiment of the present invention;

FIG. 11 is a flow chart detailing coverage of prescription products according to an exemplary embodiment of the present invention;

FIG. 12 is a flow chart illustrating how prescription products are tested for predetermined conformance according to an exemplary embodiment of the present invention; and

FIG. 13 is a block diagram illustrating an exemplary computer system for implementing an embodiment of the present invention.

### DETAILED DESCRIPTION OF THE INVENTION

Reference now will be made in detail to the presently preferred embodiments of the invention. Such embodiments are provided by way of explanation of the invention, which is not intended to be limited thereto. In fact, those of ordinary skill in the art may appreciate upon reading the present specification and viewing the present drawings that various modifications and variations can be made.

For example, features illustrated or described as part of one embodiment can be used on other embodiments to yield a still further embodiment. Additionally, certain features may be interchanged with similar devices or features not mentioned yet which perform the same or similar functions. It is therefore intended that such modifications and variations are included within the totality of the present invention.

Prior to describing the details of the invention, a brief discussion of some of the notations and nomenclature used in the description will be presented. Next, a description of exemplary hardware useable in practicing the invention will be presented.

### NOTIONS AND NOMENCLATURE

The detailed descriptions which follow may be presented in terms of program procedures executed on a computer or network of computers. These procedural descriptions and representations are the means used by those skilled in the art to most effectively convey the substance of their work to others skilled in the art.

A procedure is here, and generally, conceived to be a self-consistent sequence of steps leading to a desired result. These steps are those requiring physical manipulations of physical quantities. Usually, though not necessarily, these quantities take the form of electrical or magnetic signals capable of being stored, transferred, combined, compared and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to these quantities.

Further, the manipulations performed are often referred to in terms, such as adding or comparing, which are commonly associated with mental operations performed by a human operator. No such capability of a human operator is necessary, or desirable in most cases, in any of the operations described herein which form part of the present invention; the operations are preferably machine operations, although some or all the operations may also be manual in alternative embodiments. Useful machines for performing the operation of the present invention include general purpose digital computers or similar devices.

The present invention also relates to apparatus for performing these operations. This apparatus may be specially constructed for the required purpose or it may include a general purpose computer as selectively activated or reconfigured by a computer program stored in the computer. The procedures presented herein are not inherently related to a particular computer or other apparatus. Various general purpose machines may be used with programs written in accordance with the teachings herein, or it may prove more convenient to construct more specialized apparatus to perform the required method steps. The required structure for a variety of these machines will appear from the description given.

### CLINICAL RULES MANAGEMENT SYSTEM

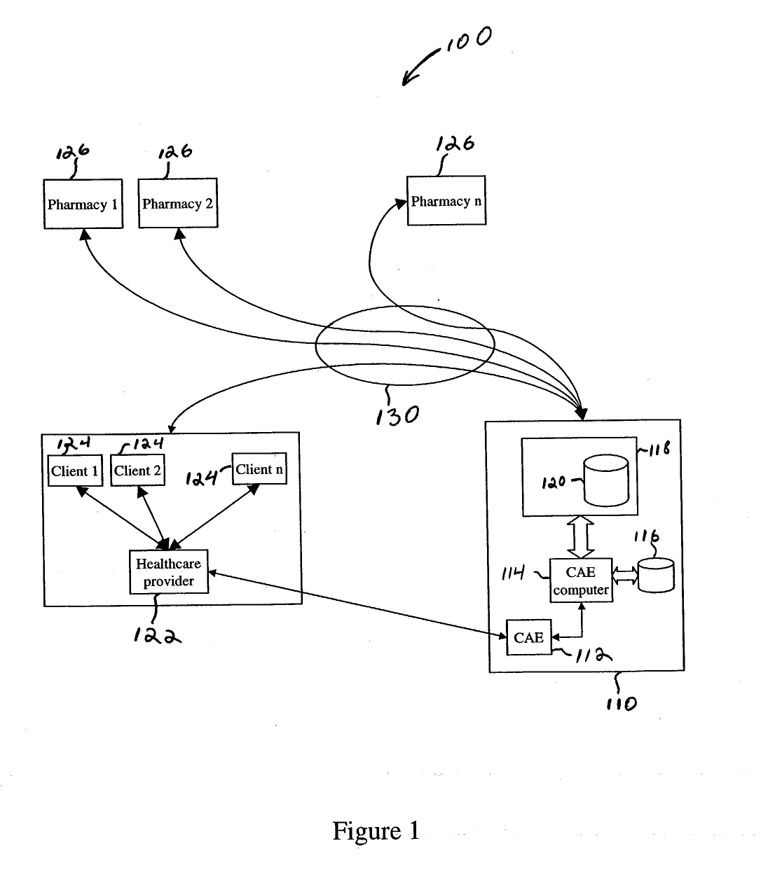

Turning now to the drawings and initially to FIG. 1, a system is shown for constructing, managing, and implementing clinical rules according to an exemplary embodiment of the present invention. The clinical rules management system, generally designated by the numeral 100, includes a prescription coverage provider 110, a healthcare provider 122, clients 124, and pharmacies 126. Although the disclosed embodiment illustrates multiple clients 124 and pharmacies 126, it should be noted that a healthcare provider 122 can support as little as one client. Additionally, it is possible to envision a system wherein a single pharmacy 126 is responsible for dispensing the prescription products to all patients. Alternatively, the prescription coverage provider 110 can include a pharmacy operating under its direct control and/or management.

The prescription coverage provider 110 can be, for example, a large pharmaceutical dispensing company responsible for filling prescription claims for the healthcare provider 122 in accordance with predefined formularies. The prescription coverage provider 110 can include, for example, a clinical account executive (CAE) 112 and a computer system 118 such as, for example, a central computer system, a networked or client-server system, etc. The CAE 112 has access to a local computer, such as a personal computer or laptop 114, that can include internal and/or external data storage devices 116 such as a fixed magnetic, digital, or optical drive. Alternatively, the external data storage device can be in the form of a database management system (not shown) for storing and/or manipulating large quantities of data.

The CAE computer 114 is capable of storing and retrieving information and/or records from the external storage device 116. In addition, the CAE computer 114 is operatively coupled to the computer system 118. More particularly, the CAE computer 114 can be part of one or more network systems which interconnect multiple computers and/or networks with each other and with the computer system 118. Thus, multiple CAEs 112 would be capable of connecting to the computer system 118 to construct and/or modify clinical rules. Such connections can include, for example, local area networks (LAN), wide area networks (WAN), wireless networks, direct dial networks (e.g., modem to modem connections), public networks (e.g., the Internet), World Wide Web, etc. Alternatively, components such as, for example, the computer system 118 and its associated data storage device 120 can be integrated into the CAE computer 114 with or without the external storage device.

Accordingly, the CAE 112 is capable of using the CAE computer 114 to access the computer 118 and exchange information from various locations. Furthermore, data retrieved from the computer 118 can be temporally and/or permanently stored on the external storage device 120. Likewise, data can be retrieved by CAE computer 114 and transmitted to the computer system 118 for storage or further manipulation and processing. It should be noted that although only one CAE 112 and associated CAE computer 114 is illustrated in FIG. 1, the present invention can provide support for multiple CAE's 112 simultaneously accessing the computer system 118 and interacting with the computer system 118 as well as other CAEs 112. Thus, the present invention should not be restricted to a single CAE 112.

As illustrated in FIG. 1, the healthcare provider 122 can be responsible for servicing the needs of multiple clients 124. The healthcare provider 122 could offer multiple prescription coverage plans from which clients 124 can choose. Optionally, the healthcare provider 122 can tailor a prescription coverage plan to each client 124, as deemed necessary. As part of the prescription coverage plan, the healthcare provider 122 and/or the clients will set some, or all, of the basic requirements of the clinical rules for accepting or covering the cost of a prescription product. Once the client's needs have been determined and/or the healthcare provider 122 has finalized the requirements of any and all prescription coverage plans, the healthcare provider 122 would typically establish a meeting with the CAE 112 to convey the requirements of the clinical rules. Such a meeting can take place in person or by other communication means such as, for example, telephone, video conferencing, web conferencing, etc. Based on the substance of this meeting, the CAE 112 would construct a plurality of clinical rules which dictate the healthcare provider's requirements for approving coverage of prescription claims.

As illustrated in FIG. 1, the healthcare provider 122 is capable of establishing a connection with the prescription coverage provider 110 over an electronic network 130. Such a connection can be established between the user computer system 126 and the computer 118 of the prescription coverage provider 110. Such a connection would typically take place across the electronic network 130, as depicted in FIG. 1. The electronic network 130 can be a public network such as the Internet or World Wide Web. Alternatively, the electronic network 130 can be a private network using private connection lines (as provided by a telephone, data, and/or wireless service provider) and/or conventional modems to establish a connection with the prescription coverage provider 110. Regardless of the type of electronic network 130 used to establish a connection, various types of protocols can be used for exchanging data between users such as, TCP/IP, FTP, Telnet, etc.

The computer 118 of the prescription coverage provider 110 stores information pertaining to prescription products. The prescription products are typically drugs (i.e., medication) and/or controlled substances that are useable for medicinal purposes and/or treatments. Such products are assigned standard specific identifiers known as a National Drug Code (NDC) identifier. New products entering the market from pharmaceutical companies must obtain government (or official) approval prior to introduction to the public. As part of this approval process, a unique NDC identifier is assigned to the pharmaceutical product. Accordingly, every pharmaceutical product that must be obtained by prescription has a unique NDC identifier. As previously indicated, the term pharmaceutical product (or product) is used to refer to any product, medication treatment, and/or therapy having an assigned NDC identifier. Currently, there are over 140,000 products assigned NDC identifiers.

As illustrated in FIG. 1, a plurality of pharmacies 126 are also capable of establishing a connection with the prescription coverage provider 110. The pharmacies 126 are capable of exchanging data with the computer system 118 in order to determine if a prescription claim should be covered, either in whole or in part. More particularly, once the clinical rules have been prepared by the CAE 112, they must be implemented as part of the healthcare provider's prescription benefits plan. Patients who require prescription products would visit a pharmacy 126 and present a prescription claim (i.e., a prescription form from a physician). However, the pharmacist cannot complete the prescription claim if it does not conform to the requirements of the healthcare provider 122. Thus, the pharmacist would contact the prescription coverage provider 110 and submit information such as the patient's account number (or similar identification number) together with the prescription product, dosage, etc. The clinical rules defined by the healthcare provider 122 for the patient's prescription benefits plan would be applied to the prescription product in order to determine if the prescription claim should be accepted or denied. Optionally, the clinical rules can dictate whether the cost of the prescription product will be covered in whole, or in part.

Every clinical rule includes various standard components such as, for example: quantity or dosage, duration of treatment, presence or absence of another prescription product which can possibly result in adverse interactions, patient history, etc. There are also various types of clinical rules such as, for example, step therapy rules, compound rules, complex rules, quantity duration rules, etc. Each specific drug and/or dosage in the clinical rule is identifiable using its assigned National Drug Code (NDC) number. Furthermore clinical rules can take various factors into account, such as the patient's age, gender, prior medical conditions, history with a particular type of prescription product, etc. Furthermore, the method of administering the prescription product, such as oral, nasal, injectible, etc., can also be taken into account. Such an arrangement allows integrated drug files (IDF) from First Databank (FDB) to be downloaded directly to the clinical rules management system to automatically update new and existing drug information. Smart keys can also be used to refer to large groups of drugs (i.e., prescription products).

A step therapy rule can be used, for example, to define guidelines for accepting certain advanced or costly prescription in view of other available options. Step therapy rules can be based on various factors, including the specific prescription product, dosage, length of treatment, etc. For example, a healthcare provider 122 may decide that certain strong pain relief prescription products will not be covered unless milder pain relief prescription products have been used. An exemplary step therapy rule could require rejection of these strong pain relief prescription products unless a milder pain relief prescription product has been prescribed within the last ninety days. Such a rule can be used to determine, in part, whether a prescribing physician has attempted less expensive treatment options for the patient. Additionally, the step therapy rule can include bypass provisions. For example, if coverage for a particular prescription product is rejected, the prescribing physician can telephone and specifically indicate that the stronger pain relief prescription product is necessary based on other diagnosed conditions.

Different healthcare providers 122 or clients 124 can have different requirements for a step therapy rule concerning strong pain relief prescription products. For example, a client 124 may request that the step therapy rule require a minimum dosage of mild pain relief prescription products before accepting the stronger prescription product. Another client 124 may require any dosage of a mild pain relief prescription product above a predetermined level before accepting the stronger prescription product. Yet another client 124 may require the use of a mild pain relief prescription product above a predetermined dosage for at least sixty of the past one hundred eighty days. It should be noted that the previous examples are in no way intended to encompass all possible options that can be implemented in the step therapy rules, but rather are intended to illustrate the flexibility with which the step therapy rules can be designed.

Compound and complex rules provide an ability to develop very focused and granular clinical rules by combining multiple decision criteria for determining whether a prescription claim will be covered. A compound or complex rule can combine single decision criteria with step therapy rules. More particularly, a compound or complex rule requires that various criteria be met before accepting a prescription. These criteria can be based on, for example, the patient, prescription product, treatment history, etc. For example, a compound or complex rule can first require that a patient be of a certain age and/or gender. Next, the patient must have a predetermined treatment history as defined, for example, by one of the previous step therapy examples. The prescription claim would only be accepted if all these criteria are met.

Compound and complex rules can also be used to identify patients with certain medical conditions in order to accept prescription products. For example, a complex rule to identify and approve prescription products for severe asthmatics can require that a determination be made to see if the patient currently receives an oral steroid. Next, a simple clinical rule determines if the oral steroid has been taken for at least fifteen of the last thirty days. Finally, if the patient also utilizes an inhaler, the complex rule would identify the patient as a severe asthmatic. In such a case, the healthcare provider 122 would accept coverage for various antihistamine products. Furthermore, the patient's history can be updated to reflect their condition as a severe asthmatic so that the same clinical rules can be bypassed in the future.

Consider, for example, a healthcare provider 122 that wants to limit the amount of brand name migraine prescription product, being prescribed because of the associated costs. A clinical committee would make a decision on how much migraine prescription product will be covered by the healthcare provider 122. The clinical committee may conclude that a thirty-day supply of prescription product is unnecessary because people generally do not have migraines every day of the month. It is possible, however, to suffer from three to five migraines within a thirty-day period. Hence, the healthcare provider 122 may request a clinical rule, for migraine prescription product, that specifies a maximum of five doses within a thirty-day period. Another healthcare provider 122 may provide a different definition for migraines, and exclude prescription products that have other benefits from the list of potential treatments. However, patients can still take the excluded prescription products to treat migraines if other conditions are met or the physician makes a special request. This provides the patient more treatment options because, for example, five dosages of an effective, brand name migraine prescription product can be used in conjunction with additional dosages of a strong pain relief prescription product to treat more severe cases.

Although FIG. 1 illustrates a single healthcare provider 122, the present invention should not be so limited. More particularly, the clinical rules management system 100 of the present invention is capable of supporting multiple healthcare providers 122. Each individual healthcare provider would also be responsible for servicing the needs of multiple clients 124. There are also situations where a physician can have capabilities for filling prescriptions for patients. In such situations, a physician computer (not shown) could be used establish a connection with the prescription coverage provider 110 and access the necessary clinical rules. Additionally, each client 124, healthcare provider 122, pharmacist 126, physician (not shown), patient (not shown), etc. can independently establish a connection with the prescription coverage provider 110 using a respective computer. Several parties (e.g., the pharmacist, physician, healthcare provide) can establish a connection with the prescription coverage provider 110 and download a copy of required clinical rules in order to further improve the efficiency with which prescription claims are covered and/or managed. In such a configuration, scheduled sessions can be established to synchronize the clinical rules stored locally at the pharmacy 126 or physician computer with a master set of clinical rules stored with the prescription coverage provider 110. Alternatively, new clinical rules can be distributed by the prescription coverage provider each time changes are made.

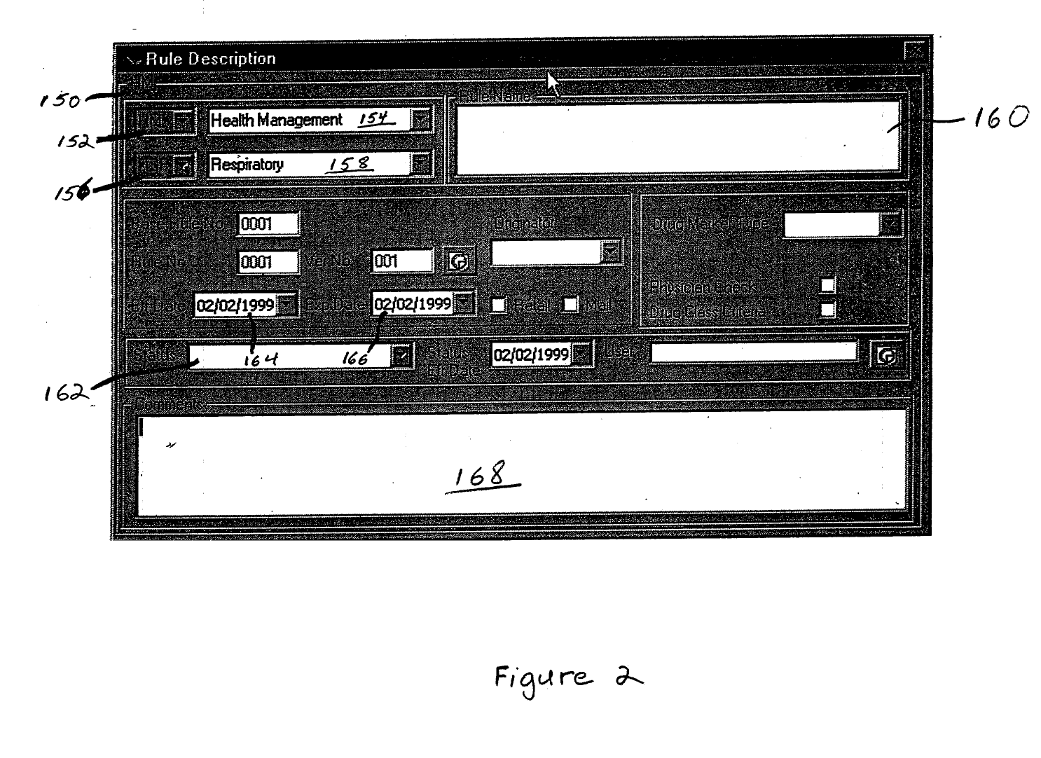

FIG. 2 illustrates a screen display for constructing (or defining) clinical rules, according to an exemplary embodiment of the present invention. Field 150 indicates the service category selected by the CAE 112, e.g., health management. The service category will typically be selected from predefined options. Field 152 is used to select a treatment category for the product. According to the illustrated embodiment of the present invention, field 152 is in the form of a conventional “drop down” menu containing predefined treatment categories that can be selected by the CAE. Alternatively, the clinical rules management system can be configured such that the CAE is given an option to independently enter a desired treatment category. This option can be provided with, or in lieu of, the predefined treatment categories. Typically, the treatment categories will be abbreviated. Field 154 can optionally displays a full description of the treatment category selected. Field 156 allows the CAE to select a treatable condition for which the prescription product can be used. This selection can be made using the illustrated drop down menu, or the CAE can be given an option to independently enter a treatable condition. Again, this option can be provided with, or in lieu of, the predefined treatable conditions. Field 158 is used to display the full description of the treatable condition when abbreviations are used. Field 160 is used by the CAE for naming and/or describing the clinical rule being defined.

The status of the clinical rule (e.g., active, inactive, suspended, etc.), can be selected by the CAE using field 162. Fields 164 and 166 are used to define the time frame in which the clinical rule will be in effect. More particularly, the CAE is capable of entering a date on which the clinical rule will become effective, as well as a date on which the clinical rule will expire. Field 168 allows the CAE to enter specific comments regarding the clinical rule and/or prescription product. According to an optional embodiment of the present invention, the comments entered by the CAE can be made available to patients, pharmacists, physicians, etc. The comments can be generally directed to the clinical rule or prescription product. For example, the comments can include negative side effects or possible adverse interactions that can occur if taken with another prescription product. The comments can also be used to educate patients on the benefits and proper use of a prescription product. Alternatively, the comments can be specifically tailored to certain healthcare providers and/or individual patients. Optionally, patients can access the clinical rules management system across an electronic network and retrieve comments regarding prescribed products.

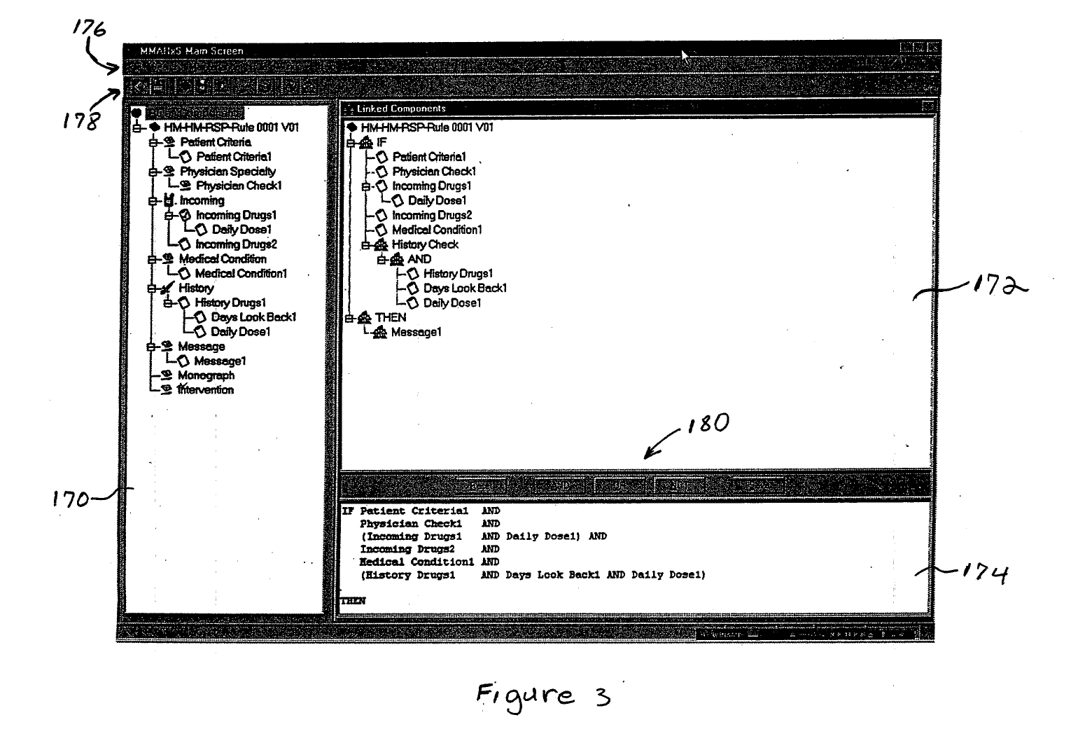

FIG. 3 illustrates a display for defining treatment types according to an exemplary embodiment of the present invention. Screen 170 provides a tree layout specifying various alternative treatment types and alternative limitations. For example, the first limitation is patient criteria which can correspond to the certain physical requirements, as will be described in greater detail below. The second limitation specifies a physician specialty, which can optionally be defined by the CAE. The physician specialty corresponds to specific fields of practice such as, for example, pediatrics, geriatrics, internal medicine, etc. The exemplary display also includes additional limitations, as previously discussed, including an “incoming” limitation which specifies the requirements of one or more incoming prescription products. The medical conditions limitation can optionally indicate special conditions of the patient and/or requirements of a prescription product. The history limitation allows the CAE to define a look back period and dosage for a particular prescription product. The CAE can also include various messages to be displayed based on which limitation is not met.

Screen 172 provides another tree layout illustrating the manner in which the limitations are processed. For example, the clinical rule requires a Boolean test (e.g. “if”) of the conditions specified in the associated branches. Additionally, the history limitation includes three sub-limitations that must be likewise tested. Depending on the result of the “if” Boolean test, a message will be displayed. The criteria for displaying the message (e.g., true or false from the “if” Boolean test), can be defined by the CAE and/or optionally predefined in the clinical rules management system. Screen 174 provides a textual display of the linked components from screen 172 in conventional Boolean query format.

According to the disclosed embodiment of the invention, clinical rules can be defined in a variety of ways using, for example, either a graphical input (or cursor control) device such as a mouse or stylus, or a conventional keyboard. The clinical rules management system can be provided with a series of “drop down” menus 176 that can be activated to reveal various command instructions for adding and/or removing limitations, selecting the details of a limitation, defining Boolean operations, etc. The menus 176 can also invoke various operations such as loading and saving various files containing the clinical rules, conducting searches for prescription products and/or clinical rules, reviewing/revising clinical rules, etc. A toolbar 178 can optionally be provided with various graphics and/or textual icons for performing various functions when activated, including some of the functions contained in the menus 176. Various additional buttons 180 can also be provided to specify the Boolean operation to be performed in screens 172 and 174 when defining the clinical rule. Thus, in creating and/or modifying a clinical rule, the CAE can simply utilize a graphical input device to add/delete limitations as well as specific criteria for a limitation. Boolean operations can be applied by marking one or more rules from screen 170 and selecting the appropriate Boolean operator. Alternatively, an input device can be used, for example, to type the details necessary to constitute the clinical rule.

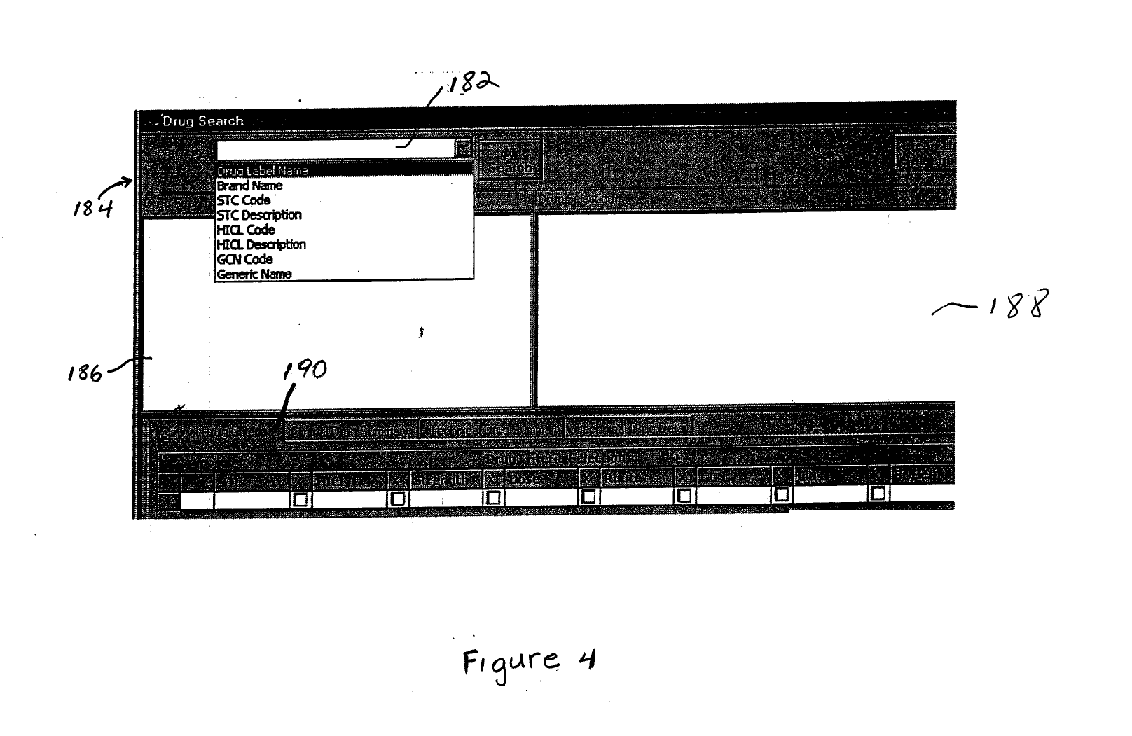

FIG. 4 illustrates a product search screen for the clinical rules management system according to an exemplary embodiment of the present invention. The product search screen allows a CAE to conduct queries for various prescription products that can be used, for example, to satisfy a clinical rule. A category field 182 is provided with a drop down menu to allow quick selection of predefined fields, or categories. A search field 184, allows the CAE to enter terms or phrases for the actual query. The search field 184 can also allow the CAE to enter Boolean operators for further refining the query. Optionally, the clinical rules management system can be designed such that a logical “or” or “and” operation is conducted on all search terms. Furthermore, an advanced search field can be provided for searching one or more categories with a single query. More particularly, the advanced search would allow formulation of a query to independently search multiple terms in different categories. Using such a configuration, the search category field 182 can be omitted or left blank.

Upon execution, the results of the query are displayed in screen 186. According to the disclosed embodiment of the present invention, the query results would generate to a list of prescription products satisfying the criteria defined by the CAE. If the query results are satisfactory, one or more prescription products can be marked and transferred to the prescription product selection screen 188. Such prescription products are then optionally applied to satisfy a particular clinical rule, or a clinical rule currently being constructed. The product search screen also includes a plurality of tabs 190 which quickly allow the CAE to bring various details regarding a marked prescription product to the forefront for review and/or analysis.

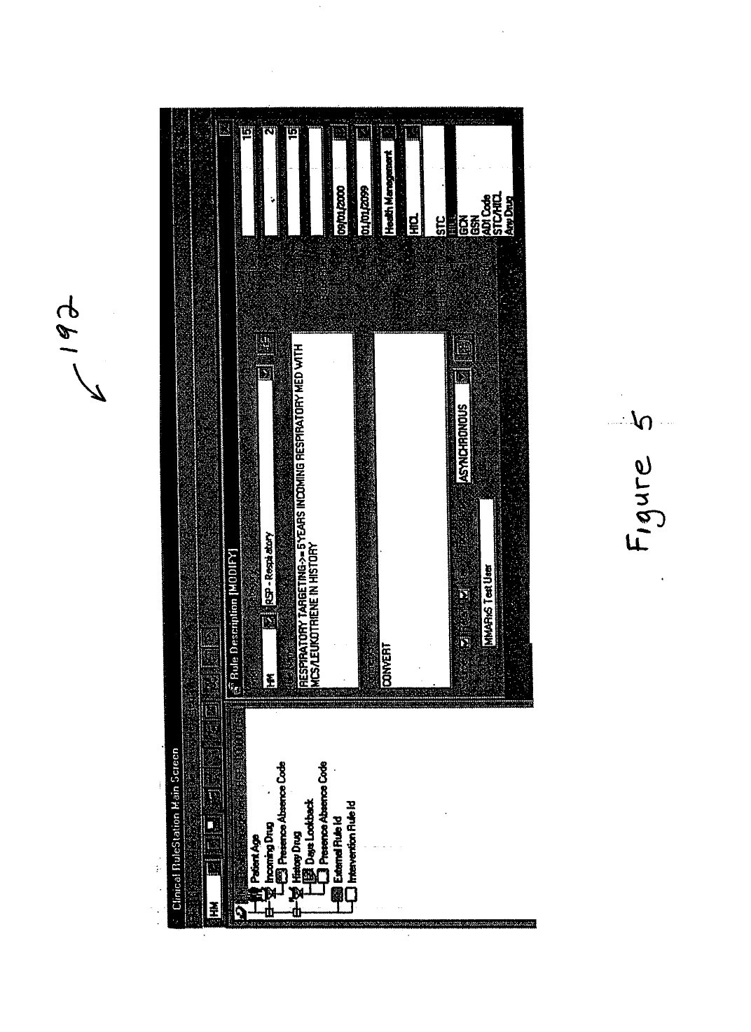

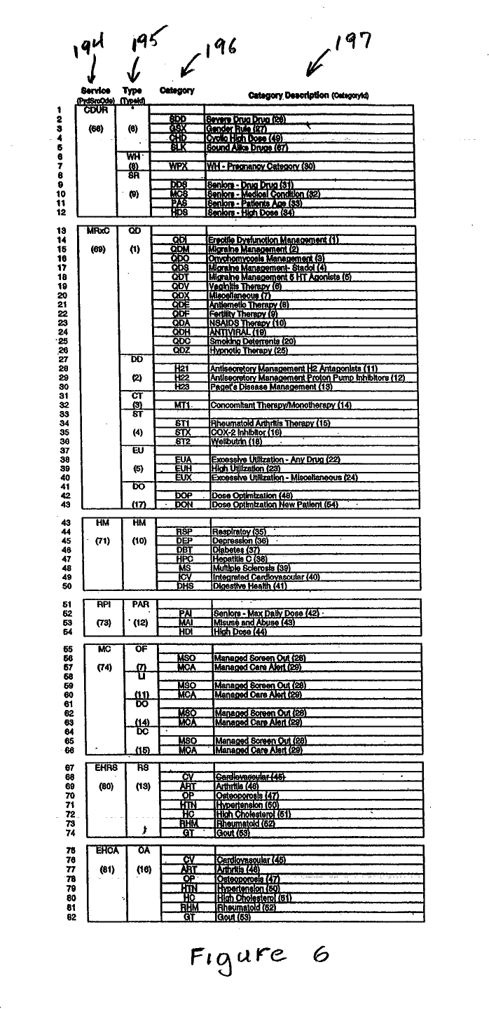

FIG. 5 illustrates a screen 192 for modifying clinical rules according to an exemplary embodiment of the present invention. Screen 192 includes most of the details shown in the rule description screen shown of FIG. 2. However, the details of the clinical rule have been pre-populated into the appropriate fields. Thus, the CAE could quickly change and/or modify the details of each field. FIG. 6 is a classification table illustrating exemplary entries for a service category 194, treatment type 195, treatable conditions 196, and category descriptions 197. The entries in the table can be predefined based on certain standard classifications, or they can be defined by the prescription coverage provider.

### CREATING CLINICAL RULES

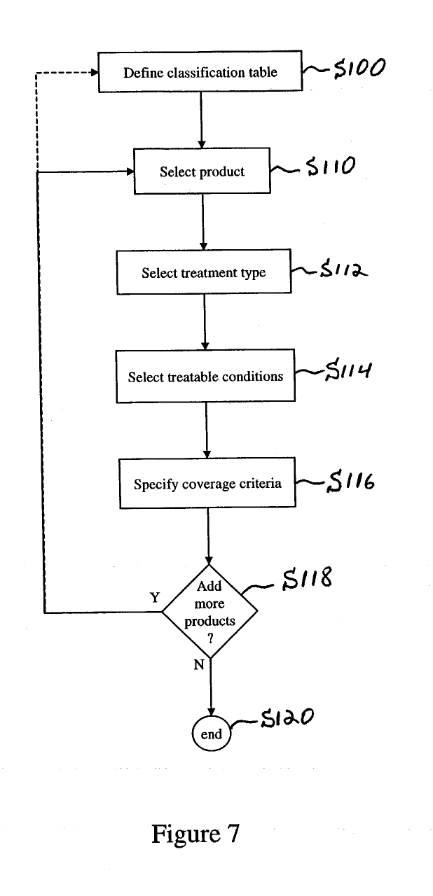

Turning now to FIG. 7, a flowchart is illustrated for generally outlining the steps performed in defining clinical rules, according to an exemplary embodiment of the present invention. At step S100, the service classification table is defined. This typically entails, for example, the CAE creating a table, such as the classification table illustrated in FIG. 6. The classification table contains entries for various columns, including a service category, a treatment category, a treatable condition category, and a category description. Optionally, the classification table is predefined by the prescription coverage provider, thus eliminating step S100 and the need to create one while defining the clinical rules.

At step S110, a prescription product is selected. As previously discussed, the prescription product can be a specific prescription product and/or treatment for which the healthcare provider will accept coverage. At step S112, a treatment type is selected. The treatment type generally corresponds to the manner in which the prescription product will be used by the patient. For example, the treatment type could be a quantity duration treatment, a step therapy treatment, a dosage duration treatment, etc. The treatment type corresponds to the type column in FIG. 6. At step S114, various treatable conditions are selected. The treatable conditions correspond to entries listed in the category column of FIG. 6. For example, the treatable conditions, can be used to identify treatment categories such as, for example, smoking, anti-viral, migraine, respiratory, depression, diabetes, etc.

At step S116, the specific criteria for covering the prescription product is entered by the CAE. The coverage criteria include the details of the treatment type that will be applied to determine whether the prescription product should be covered. More particularly, the coverage criteria would correspond to details of the clinical rule as requested by the healthcare provider and/or limited by the prescription coverage provider. In a quantity duration treatment, for example, the coverage criteria may specify a maximum dosage and frequency for taking a prescription product within a specified length of time. At step S118, it is determined if additional prescription products will be added to the rule. Alternatively, such a step can correspond to determining whether a new clinical rule will be defined. If additional products will be added, then control returns to step S110. In the event that a new clinical rule will be defined, control would optionally return to step S100. If no additional products will be added, then control passes to step S120 where the process ends.

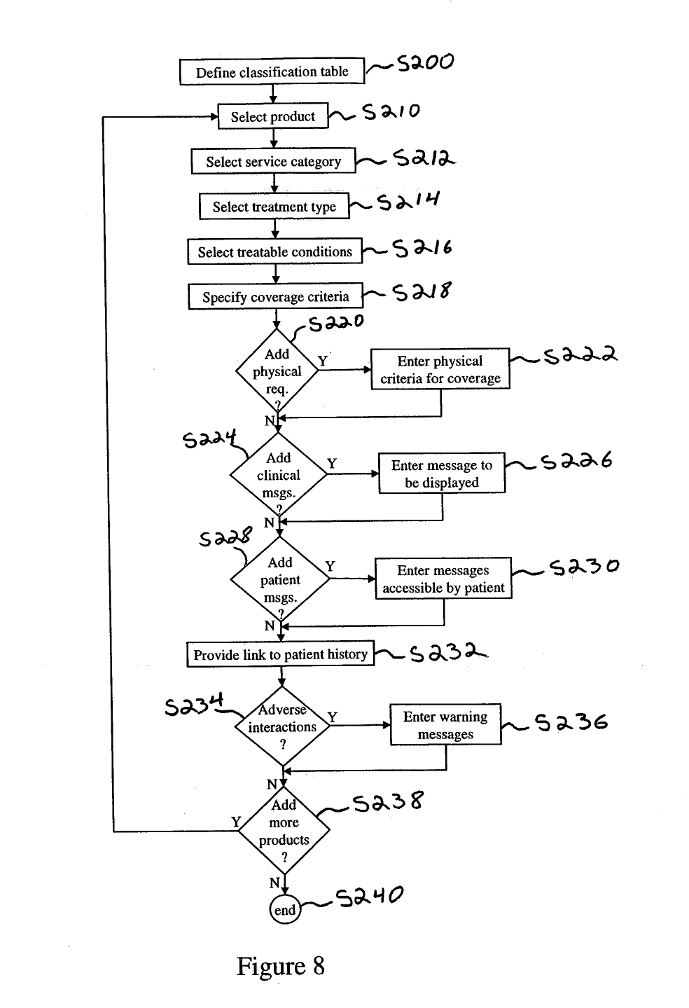

FIG. 8 is a flowchart outlining, in further detail, the steps performed in defining clinical rules. At step S200, the service classification is selected by the CAE. At step S210, the prescription product for which the rule will be applied is selected. At step S212, a service category is selected for the product. At step S214, the treatment type is selected. At step S216, the treatable conditions for which the clinical rule will be applicable are selected. At step S218, the specific criteria for providing coverage of the prescription product are specified. At step S220, it is determined if certain physical requirements should be reviewed prior to approving coverage of a prescription product. The physical requirements can correspond to features such as, for example, the patient's age, gender, weight, etc., that must be considered in conjunction with the prescription product. If physical requirements will be added to the clinical rule, then at step S222, the CAE would enter the details for the physical criteria recovered for coverage of the prescription product.

If no physical requirements are going to be added, then control passes to step S224, where it is determined if clinical messages will be added to the clinical rule. If so, then at step S226, the CAE would input the clinical messages that can be displayed when a patient attempts to obtain coverage of the specified prescription product. If no clinical messages will be added, then control passes to step S228. At step S228, it is determined if patient messages will be added to the clinical rule. If such is the case, then control passes to step S230 where the messages are entered. Such messages can, for example, correspond to, various warnings regarding the benefits and side effects of the prescription product. Various other messages can be included that are intended to educate the patient regarding the prescription product. If the CAE will not include patient messages, then control passes to step S232.

At step S232, a link is provided to the patient's clinical history in order to determine whether adverse reactions could possibly result from taking the current prescription product concurrently with other prescription products that the patient may also be taking. Additionally, a comparison can be made to assess the patient's drug utilization history in order to determine whether the same prescription product or similar prescription products have previously been rejected because of special medical conditions from which the patient suffers. At step S234, it is determined if adverse interactions will occur by taking the current prescription product with products in the patient's medical clinical history. Various messages are entered, at step S236, in the event that adverse interactions will occur. If no adverse interactions will occur, then control passes to step S238, where it is determined if additional products will be added to the clinical rule. If additional products will be added, then control returns to step S210. As previously indicated, however, control can optionally return to step S210 in order to create an entirely new clinical rule. If no additional products will be added, then control passes to step S240 where the process ends.

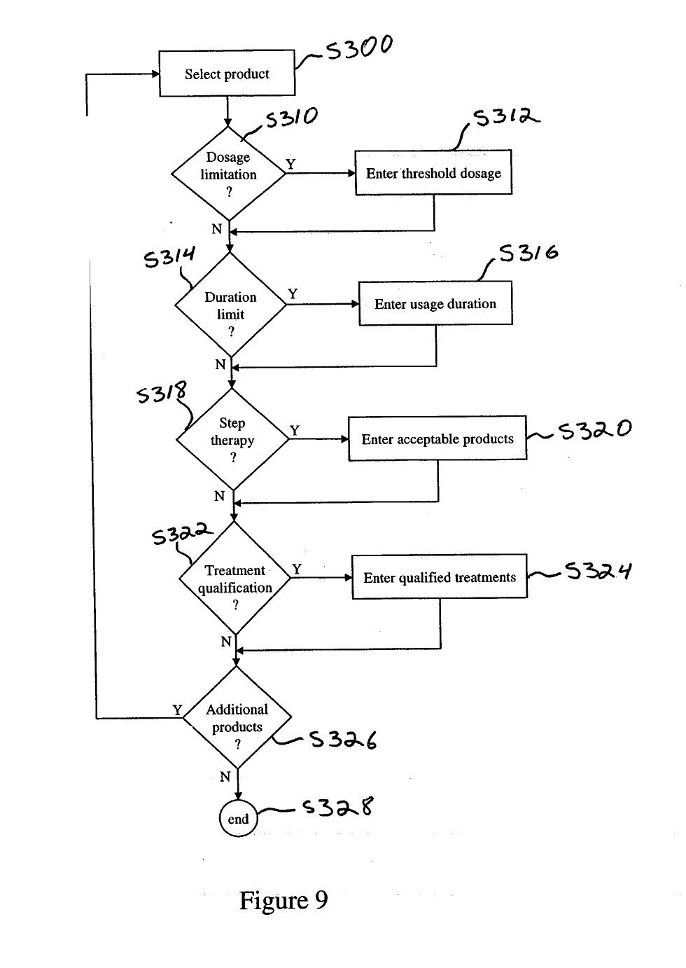

FIG. 9 is a flowchart illustrating the details of selecting specific treatment types for clinical rules according to an exemplary embodiment of the present invention. The process begins at step S300, where a particular prescription product is selected. At step S310, it is determined if a dosage limitation should be imposed on the prescription product as part of the clinical rule. If so, then at step S312, the CAE would enter the threshold dosage for the product. Such a threshold dosage can, for example, correspond to a minimum quantity, a maximum quantity, or equivalent quantities based on the specific method for delivering the prescription product. If no dosage limitation will be included, then control passes to step S314. It is then determined if a duration limit will be imposed on the prescription product. If so, control passes to step S316 where the CAE inputs the details of the duration for using the prescription product. For example, the duration limit can correspond to a maximum length of time for using the prescription product, a maximum number of dosages allowed within a predetermined time period, etc.

Control passes to step S318 if a duration limit is not entered. At step S318, it is determined if a step therapy treatment is in order. If so, control passes to step S320, where the CAE would enter the particulars of acceptable products and dosages that can be used as part of the step therapy. A step therapy treatment, for example, can correspond to a requirement that the patient has used other products having a milder dose or type prior to using the current prescription product. Control passes to step S322, if a step therapy treatment will not be included in the rule. At step S322, it is determined if a treatment history qualification will be included in the rule. If so, the CAE would enter the qualified treatment types at step S324. A treatment history qualification would specify the prescription products, or treatments, that should be present in the patient's clinical history in order to justify approval of the prescription product. At step S326, it is determined if additional products are available for specifying the treatment types. If so, then control returns to step S300. Otherwise, then the process ends at step S328.

### PROCESSING CLINICAL RULES

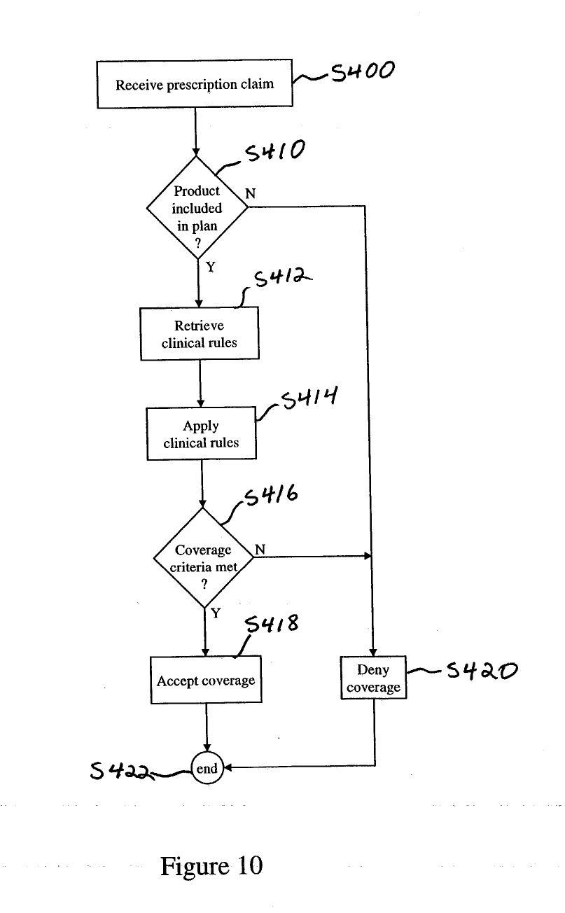

FIG. 10 is a flowchart illustrating the steps performed when applying a clinical rule to determine if a prescription claim should be accepted. At step S400, the prescription claim is received, for example, at the pharmacy. Typically, a patient would obtain a prescription from a physician, or other authorized medical personnel, and submit the prescription to a pharmacist to be filled. The prescription would typically include one or more prescription products and directions for taking the products. The pharmacist would subsequently input the prescription information into a local computer. The prescription information would be transmitted to the clinical rules management system for application of various clinical rules. The clinical rules management system would then indicate if the cost of the prescription will be covered and, optionally, to what degree.

At step S410, it is determined if one or more of the products in the prescription claim are included in the patient's prescription benefits plan. If the products are not included, then control would pass to step S420 and coverage of the prescription claim would be denied. If the prescription product is included in the patient's prescription benefits plan, then various clinical rules are retrieved at step S412. At step S414, the clinical rules are applied to the prescription product. At step S416, it is determined if the coverage criteria for the prescription product has been met. More particularly, the various requirements set forth in the clinical rules are processed in order to see if the product being prescribed, and the dosage levels, satisfy the requirements set forth by the healthcare provider. If the coverage criteria are not met, then control passes to S420 and coverage is denied. If the coverage criteria are met, then control passes to step S418 where coverage of the prescription product is accepted. At step S420, the process ends.

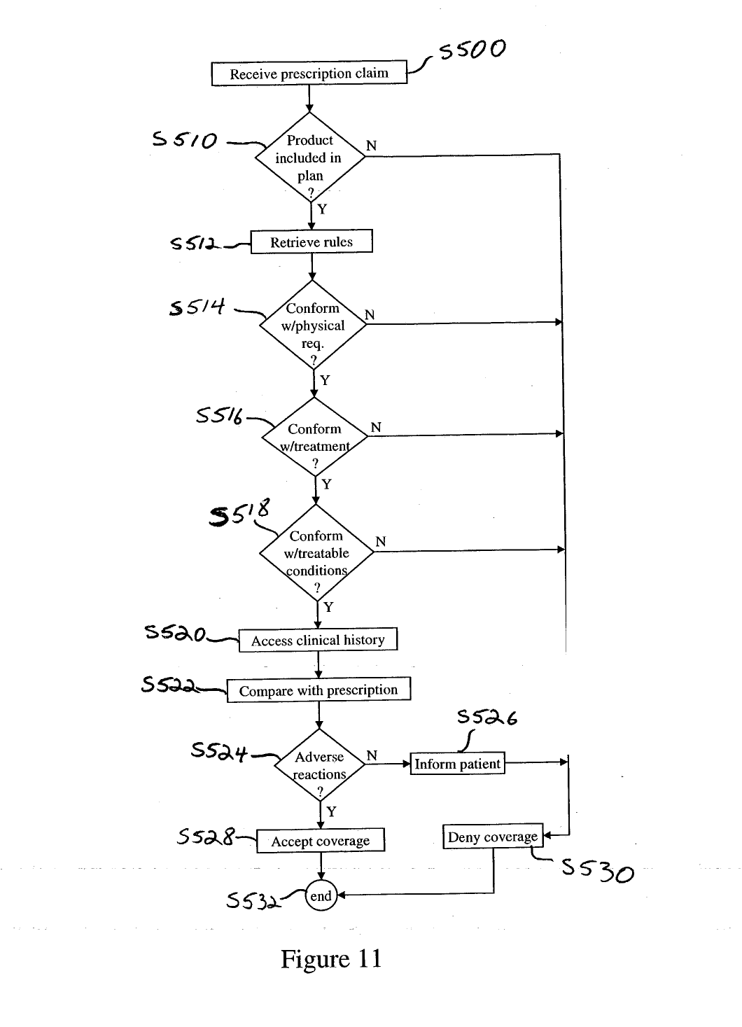

FIG. 11 is a flowchart illustrating, in further details, the manner in which coverage of prescription products are determined. At step S500, the prescription claim is received. At step S510, it is determined if products from the prescription claim are included in the patient's prescription benefits plan. If the products are not included in the patient's prescription benefit plan, then control passes to step S530 and coverage of the prescription claim is denied. If the products are included in the patient's prescription plan, then at step S512, the appropriate clinical rules are retrieved and applied to the prescription product. At step S514, it is determined if the prescription product and/or patient conform to the physical requirements of the clinical rule. For example, if a particular prescription product is not suited for children under the age of 12, then the patient's age would be checked to ensure that the patient is at least 13 years of age. Likewise, a prescription product having unacceptable effects on a patient of a certain gender would not conform to the physical requirement. Accordingly, control would pass to step S530, where the prescription claim would be denied coverage.

If the prescription product conforms to the physical requirements set forth in the clinical rules, then control passes to step S516. At step S516, it is determined if the prescription product and/or the instructions included in the prescription claim (or the actual prescription product's packaging) conform to the treatment limitations set forth by the healthcare provider. If they do not conform, then control passes to step S530 where coverage of the prescription is denied. If the instructions from the prescription claim conform to the requirements of the healthcare provider, as set forth in the clinical rules, then control passes to step S518. At step S518, it is determined if the illness for which the prescription product is being used conforms to the treatable conditions specified by the healthcare provider. For example, a physician may prescribe a particular pain reliever to a patient suffering from migraine headaches. However, the prescription product may not be supported by the clinical rules and/or the formulary covered by the healthcare provider. In such a case, the physician would have to prescribe an alternative pain killer that is capable of treating migraine headaches. Accordingly, the prescription product would not conform to the treatable conditions, and the control would pass to step S530 where coverage would be denied.

It should be noted that, optionally, the pharmacist may contact the clinical rules management system (or a CAE) and request a substitute product that can be suitably used by the patient in order to avoid denying coverage of the prescription claim and requiring a subsequent visit to the physician. At step S520, the patient's clinical history is accessed. More particularly, as the prescription claim is being processed, clinical data from the patient's file is automatically received from the database server, or other storage device, by the computer system. Data contained in the retrieved clinical history would be compared to the prescription products at step S522. Based on this comparison, it is determined, at step S524 if adverse reactions would occur from the use of the prescription product. If adverse reactions would occur, then at step S526, the pharmacist would inform the patient. Control would then pass to S530, where coverage of the prescription claim would be denied. If there are no adverse reactions, then control passes to step S528 where coverage for the prescription product is accepted. Optionally, upon informing the patient of adverse reactions, the pharmacist may contact and advice the patient's physician to see if a revise prescription can be prepared. The process ends at step S532.

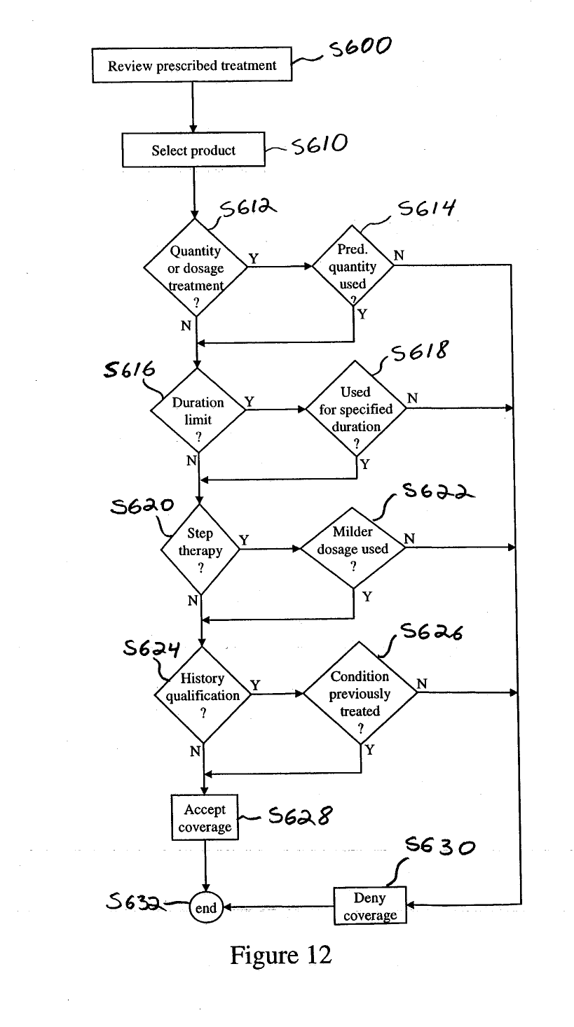

FIG. 12 is a flowchart illustrating the details for determining whether the prescription product conforms to the treatment requirements of the healthcare provider. At step S600, the prescribed treatment is reviewed by the prescription coverage provider. In other words, the instructions provided by the physician on the prescription claim are reviewed. At step S610, the product is selected. At step S612, the clinical rules management system determines if a quantity, or dosage, treatment rule should be applied to the prescription product. If so, then at step S614, the clinical rules management system examines the prescribed treatment to see if a predetermined quantity (or dosage) has been prescribed by the physician. For example, if the healthcare provider requests a clinical rule that limits the amount of migraine prescription product to six dosages per month, then a prescription claim requiring ten dosages of the migraine prescription product would not satisfy the predetermined quantity limitation. Control would then pass to step S630 where coverage is denied. Otherwise, control passes to step S616. Control would also pass to step S616, if there is no quantity treatment.

At step S616, it is determined if a duration limit has been imposed on the prescription product by the clinical rules. If there is no duration limit, then control passes to step S620. Otherwise, control passes to step S618, where it is determined if use of the prescription product will be used for the specified duration limit. If use of the prescription product will exceed, or otherwise conflict with, the clinical rule, then control passes to step S630 where coverage of the prescription claim is denied. If the duration limits are satisfied, the control passes to step S620.

At step S620, the clinical rules management system determines if a step therapy is included in the prescription (e.g. the prescribed treatment). If so, then at step S622 it is determined if a milder dosage of the prescription product (or similar products) has previously been prescribed to the patient. If milder products have not been prescribed, then control passes to step S630 and coverage is denied. If milder products have been prescribed, then control passes to step S624. Likewise, control would also pass to step S624 if no step therapy was included in the prescribed treatment. At step S624, the clinical rules management system determines if a treatment history qualification is included in the clinical rule. If so, then at step S626, it is determined if the patient has previously been treated for the same medical condition. If not, then control passes to step S630, and coverage is denied. If the patient has been treated for the same medical condition, then control passes to step S628. The prescription claim would then be accepted. The process ends at step S632.

### HARDWARE OVERVIEW

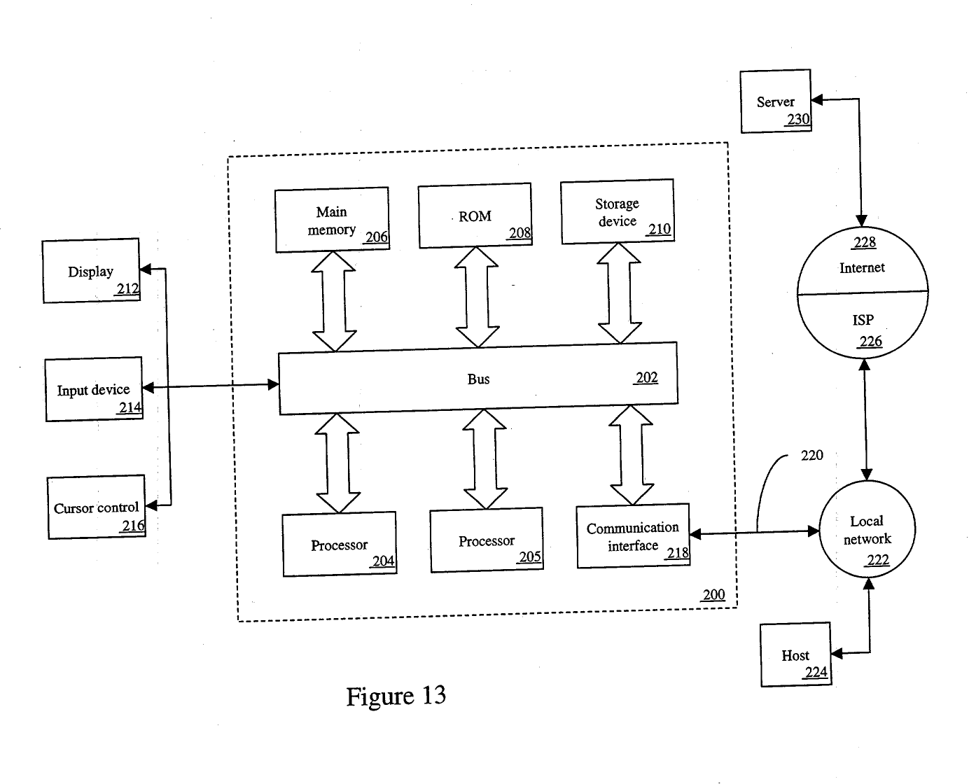

FIG. 13 is a block diagram that illustrates a computer system 200 upon which an embodiment of the invention may be implemented. Computer system 200 includes a bus 202 or other communication mechanism for communicating information, and a processor 204 coupled with bus 202 for processing information. Computer system 200 also includes a main memory 206, such as a random access memory (RAM) or other dynamic storage device, coupled to bus 202 for storing information and instructions to be executed by processor 204. Main memory 206 also may be used for storing temporary variables or other intermediate information during execution of instructions to be executed by processor 204. Computer system 200 further includes a read only memory (ROM) 208 or other static storage device coupled to bus 202 for storing static information and instructions for processor 204. A storage device 210, such as a magnetic disk or optical disk, is provided and coupled to bus 202 for storing information and instructions.

Computer system 200 may be coupled via bus 202 to a display 212, such as a cathode ray tube (CRT), for displaying information to a computer user. An input device 214, including alphanumeric and other keys, is coupled to bus 202 for communicating information and command selections to processor 204. Another type of user input device is cursor control 216, such as a mouse, a trackball, or cursor direction keys for communicating direction information and command selections to processor 204 and for controlling cursor movement on display 212. This input device typically has two degrees of freedom in two axes, a first axis (e.g., x) and a second axis (e.g., y), that allows the device to specify positions in a plane.

The invention is related to the use of computer system 200 for constructing and managing clinical rules. According to one embodiment of the invention, construction of clinical rules is performed by computer system 200 in response to processor 204 executing one or more sequences of one or more instructions contained in main memory 206. Such instructions may be read into main memory 206 from another computer-readable medium, such as storage device 210. Execution of the sequences of instructions contained in main memory 206 causes processor 204 to perform the process steps described herein. One or more processors in a multi-processing arrangement may also be employed to execute the sequences of instructions contained in main memory 206. In alternative embodiments, hard-wired circuitry may be used in place of or in combination with software instructions to implement the invention. Thus, embodiments of the invention are not limited to any specific combination of hardware circuitry and software.

The term “computer-readable medium” as used herein refers to any medium that participates in providing instructions to processor 204 for execution. Such a medium may take many forms, including but not limited to, non-volatile media, volatile media, and transmission media. Non-volatile media include, for example, optical or magnetic disks, such as storage device 210. Volatile media include dynamic memory, such as main memory 206. Transmission media include coaxial cables, copper wire and fiber optics, including the wires that comprise bus 202. Transmission media can also take the form of acoustic or light waves, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 204 for execution. For example, the instructions may initially be borne on a magnetic disk of a remote computer. The remote computer can load the instructions into its dynamic memory and send the instructions over a telephone line using a modem. A modem local to computer system 200 can receive the data on the telephone line and use an infrared transmitter to convert the data to an infrared signal. An infrared detector coupled to bus 202 can receive the data carried in the infrared signal and place the data on bus 202. Bus 202 carries the data to main memory 206, from which processor 204 retrieves and executes the instructions. The instructions received by main memory 206 may optionally be stored on storage device 210 either before or after execution by processor 204.

Computer system 200 also includes a communication interface 218 coupled to bus 202. Communication interface 218 provides a two-way data communication coupling to a network link 220 that is connected to a local network 222. For example, communication interface 218 may be an integrated services digital network (ISDN) card or a modem to provide a data communication connection to a corresponding type of telephone line. As another example, communication interface 218 may be a local area network (LAN) card to provide a data communication connection to a compatible LAN. Wireless links may also be implemented. In any such implementation, communication interface 218 sends and receives electrical, electromagnetic or optical signals that carry digital data streams representing various types of information.

Network link 220 typically provides data communication through one or more networks to other data devices. For example, network link 220 may provide a connection through local network 222 to a host computer 224 or to data equipment operated by an Internet Service Provider (ISP) 226. ISP 226 in turn provides data communication services through the worldwide packet data communication network, now commonly referred to as the “Internet” 228. Local network 222 and Internet 228 both use electrical, electromagnetic or optical signals that carry digital data streams. The signals through the various networks and the signals on network link 220 and through communication interface 218, which carry the digital data to and from computer system 200, are exemplary forms of carrier waves transporting the information.

Computer system 200 can send messages and receive data, including program code, through the network(s), network link 220, and communication interface 218. In the Internet example, a server 230 might transmit a requested code for an application program through Internet 228, ISP 226, local network 222 and communication interface 218. In accordance with the invention, one such downloaded application provides an ability for patients to access various messages regarding prescription products described herein. The received code may be executed by processor 204 as it is received, and/or stored in storage device 210, or other non-volatile storage for later execution. In this manner, computer system 200 may obtain application code in the form of a carrier wave.

Major objectives and advantages of the present invention are convenience and cost reduction (where appropriate, safe, and effective). The clinical rules management system stands to benefit healthcare providers, physicians, pharmacists, and patients. More particularly, healthcare providers are capable of providing patients with more options for their prescription benefits plan, because the amount of time required to define clinical rules can be significantly reduced. The overall cost of the prescription benefits plan can also be reduced because less time is required to create the clinical rules. Patients benefit from increased options and reduced costs. Patients also benefit from increased efficiency achieved by the clinical rules management system. More particularly, prescription claims can be processed with greater speed and accuracy. Thus, inappropriate coverage denials can be reduced. Furthermore, costs are reduced because of the improved efficiency realized by the prescription coverage provider and pharmacist.

Rather than programming individual subroutines, modules, or applets for each clinical rule, various embodiments of the present clinical rules management system can define individual clinical rules in terms of various attributes. These attributes can be stored in a database server where they can be quickly queried and/or modified. Clinical rules can be created by simply selecting various attributes and/or inputting a range of values such as, for example, an age range. Such a configuration can significantly reduce the amount of time required to create a clinical rule.

The present clinical rules management system is optionally used to create a set of simple and complex coverage and drug utilization rules (DUR) that are tailored specifically for each client supported by every healthcare provider. A product code can be used to identify which set of DURs is associated with each healthcare provider. When a prescription claim is received, the healthcare provider profile can be immediately associated with the claim in order to determine whether the prescription product should be covered.

The present clinical rules management system is optionally configured to provide a processing hierarchy for each clinical rule. More particularly, when using complex rules, for example, the clinical rules management system will process the individual clinical rules on a first-in-first-out basis. The first clinical rule encountered will be processed and if a termination condition is reached then the prescription product will not be covered. Otherwise, the next clinical rule will be processed. This process continues until either all clinical rules have been applied, or a termination condition is reached.

As previously discussed, the present clinical rules management system is configured to include a computer system and appropriate communication hardware and software to establish a connection over a public or private communication network. Such networks can include; for example, the World Wide Web, the Internet, Local Area Networks (LANs), Wide Area Networks (WANs), direct-dial telephone networks, etc. The clinical rules management system can be accessed by pharmacists, physicians, patients, healthcare providers, etc. over the communication network. The clinical rules management system can then be used to obtain information regarding specific clinical rules for a healthcare provider, prescription product, etc. Additionally, a distributed computer system can be used by the prescription coverage provider to maintain and control access to the clinical rules. For example, in systems such as a client-server arrangement, various aspects of the clinical rules and database server can be stored on different servers that are interconnected by the communication network for exchanging information. Additionally, the servers can function as backup, or redundant, systems for emergency situations.

Various access rights can be defined depending on who accesses the clinical rules management system. As previously indicated, one or more product codes can be assigned to each healthcare provider. The product code can be used, in part, to identify the clinical rules associated with a particular client. Accordingly, the access rights can be based, in part, on the product code. Such a configuration would allow a healthcare provider to access information regarding their own clinical rules, without compromising privacy rights of other healthcare providers. Similarly, physicians and pharmacists can access the information required to either write or fill a prescription based on patient identification numbers assigned by the healthcare provider and/or client. Such identification numbers can be associated with the product code for the client in order to determine whether a particular prescription product will be covered for the patient. Patients can also access the clinical rules management system in order to retrieve information regarding prescription coverage or specific prescription products.

The clinical rules management system is optionally configured such that the information accessible over the communication network is tailored specifically for different users. For example, the healthcare provider can obtain any information regarding coverage, prescription product, or clinical rules for the client. The physician and pharmacist can obtain standard National Council for Prescription Drug Programs (NCPDP) guidelines for exchanging and processing prescription information. Physicians and pharmacists can also receive messages indicating why a particular prescription product will not be covered. In such circumstances, it is possible to contact an administrator, or CAE, to discuss the need for the prescription product. Information viewed by a patient would differ from the information viewed by a physician, because patients typically do not understand the medical terminology used by pharmacists and physician. Accordingly, patients would be provided with extensive explanations of things such as prescription coverage, medication benefits and warnings, drug interactions, required dosage, etc.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A method of creating clinical rules for approving coverage of prescription products comprising the steps:
selecting at least one service classification for a clinical rule;
selecting at least one prescription product for the clinical rule;
selecting at least one treatment type for each selected prescription product;
selecting predetermined medical conditions treatable by the prescription products under each clinical rule, based on the defined service classifications; and
specifying at least one coverage criteria for approving coverage of each selected prescription product.

2. The method of claim 1, further comprising inputting clinical messages regarding the prescription product for each clinical rule.

3. The method of claim 2, wherein the clinical messages provide information regarding coverage limits for the prescription product.

4. The method of claim 2, wherein different clinical messages are provided for retail pharmacies and mail order pharmacies.

5. The method of claim 1, further comprising inputting patient messages regarding the prescription product for each coverage criteria.

6. The method of claim 5, further comprising the steps:
establishing a communication channel with a patient computer; and
providing access to the patient messages across the communication channel.

7. The method of claim 1, further comprising selecting predetermined physical criteria to support coverage of the prescription product.

8. The method of claim 7, wherein selecting predetermined physical criteria includes a step of selecting at least one of gender, age, and weight.

9. The method of claim 1, further comprising inputting adverse interactions that can result from using the prescription product.

10. The method of claim 9, wherein inputting includes a step of providing a link to a patient's prescription history to identify prescription products that have been, or are currently, used by the patient.

11. The method of claim 10, further comprising defining a predetermined time interval within the patient's clinical history to be reviewed.

12. The method of claim 1, further comprising defining at least one delivery method for administering the prescription products.

13. The method of claim 12, further comprising calculating equivalent dosages of the prescription products for different delivery methods.

14. The method of claim 1, wherein the step of selecting at least one service classification further includes selecting a service category and a treatable condition for each selected service classification.

15. The method of claim 1, wherein the step of selecting at least one treatment type includes a step of selecting a combination of one or more treatment types from the group of treatment types consisting of quantity, duration, step therapy, and treatment history qualification.

16. The method of claim 15, wherein the treatment type is a quantity treatment, and further comprising the steps:
providing a link to the patient's clinical history; and
specifying, in the coverage criteria, that the prescription product should be covered only if the patient has previously used a predetermined quantity of the same prescription product for the medical condition.

17. The method of claim 15, wherein the treatment type is a duration treatment, and further comprising the steps:
providing a link to the patient's clinical history; and
specifying, in the coverage criteria, that the prescription product should be covered only if the patient has previously used the same prescription product for a predetermined length of time for the same medical condition.

18. The method of claim 15, wherein the treatment type is a step therapy treatment, and further comprising the steps:
providing a link to the patient's clinical history; and
specifying, in the coverage criteria, that the prescription product should be covered only if the patient has previously used a milder prescription product for the same medical condition.

19. The method of claim 15, wherein the treatment type is a treatment history qualification, and further comprising the steps:
providing a link to the patient's clinical history; and
specifying, in the coverage criteria, that the prescription product should be covered only if the patient's clinical history indicates a pattern of predetermined treatment types prescription product for the same medical condition.

20. The method of claim 15, further comprising the steps:
selecting multiple treatment types; and
linking selected treatment types to a single coverage criteria.

21. The method of claim 1, further comprising the steps of providing an operator with a graphical interface, and wherein at least one of the steps of defining, selecting at least one treatment type, selecting predetermined medical conditions, and identifying is performed using an input device.

22. The method of claim 1, further comprising the steps of providing an operator with a graphical interface, and wherein at least one of the steps of defining, selecting at least one treatment type, selecting predetermined medical conditions, and identifying is performed using a graphical/cursor control device.

23. A method of evaluating prescription claims using predefined clinical rules, comprising the steps:
determining whether at least one prescription product in a prescription claim are included in a prescription benefits plan of at least one client;
retrieving a plurality of clinical rules from the prescription benefit plan; and
applying at least one of the plurality of clinical rules to the at least one prescription product to determine whether the prescription claim is reimbursable, at least in part, with respect to the at least one prescription product.

24. The method of claim 23, wherein the step of applying further includes a step of comparing physical criteria contained in the clinical rule with patient criteria.

25. The method of claim 23, wherein the step of applying further includes a step of comparing treatment types contained in the clinical rules with a treatment type specified in the prescription claim.

26. The method of claim 23, wherein the step of applying further comprises a step of comparing medical conditions contained in the clinical rules with a medical condition specified in the prescription claim.

27. The method of claim 23, wherein the step of applying further comprises the steps:
comparing prescription products in the patients clinical history with the prescription products in the prescription claim; and
identifying adverse reactions that can result from using the prescription products in the patient's clinical history simultaneously with the prescription products in the prescription claim.

28. The method of claim 23, further comprising a step of determining a treatment type for each prescription product in the prescription claim.

29. The method of claim 28, wherein the treatment type is a quantity treatment, and further comprising the steps:
reviewing the patient's clinical history; and
accepting coverage for the prescription product only if the patient has previously used a predetermined quantity of the same prescription product for the same medical condition.

30. The method of claim 28, wherein the treatment type is a duration treatment, and further comprising the steps:
reviewing the patient's clinical history; and
accepting coverage for the prescription product only if the patient has previously used the same prescription product for a predetermined length of time for the same medical condition.

31. The method of claim 28, wherein the treatment type is a step therapy treatment, and further comprising the steps:
reviewing the patient's clinical history; and
accepting coverage for the prescription product only if the patient has previously used a milder prescription product for the same medical condition.

32. The method of claim 28, wherein the treatment type is a treatment history qualification, and further comprising the steps:
reviewing the patient's clinical history; and
accepting coverage for the prescription product only if the patient's clinical history indicates a pattern of predetermined treatment types prescription product for the same medical condition.

33. The method of claim 23, further comprising a step of assessing the severity of a patient's medical condition.

34. The method of claim 33, wherein the step of assessing further comprises the steps:
reviewing the patient's clinical history to identify prescription products which have been prescribed to the patient;
determining the medical conditions that are treatable by identified prescription products; and
identifying one or more correlations between the medical conditions and prescription products.

35. The method of claim 34, further comprising a step of identifying trends in dosage of a prescription product taken by a patient for one of the determined medical conditions.

36. A method of creating clinical rules for approving coverage of prescription products comprising the steps:
selecting at least one service classification for each of a plurality of clinical rules;
selecting at least one prescription product for the at least one of the plurality of clinical rules;
selecting at least one treatment type for each selected prescription product;
selecting predetermined medical conditions treatable by the prescription products under each of the plurality of clinical rules, based on the defined service classifications;
specifying at least one coverage criteria for approving coverage of each selected prescription product;
selecting predetermined physical criteria to support coverage of the prescription product; and
inputting clinical messages regarding the prescription product for each clinical rule.

37. A method of creating clinical rules for approving coverage of prescription products comprising the steps:
selecting at least one service classification for a clinical rule;
selecting at least one prescription product for the clinical rule;
selecting a combination of one or more treatment types for each selected product from the group of treatment types consisting of quantity, duration, step therapy, and treatment history qualification;
selecting predetermined medical conditions treatable by the prescription products under each clinical rule, based on the defined service classifications;
specifying at least one coverage criteria for approving coverage of each selected prescription product; and
defining at least one delivery method for administering the prescription products.

38. A method of evaluating prescription claims using predefined clinical rules, comprising the steps:
determining if one or more prescription products in a prescription claim are included in a client's prescription benefits plan;
retrieving a plurality of clinical rules from the client's prescription benefit plan;
determining a treatment type for each prescription product in the prescription claim;
applying an appropriate clinical rule to each prescription product to determine if the prescription claim should be covered, at least in part, with respect to the prescription products; and
assessing the severity of a patient's medical condition.

39. A method of evaluating prescription claims using predefined clinical rules, comprising the steps:
determining if one or more prescription products in a prescription claim are included in a client's prescription benefits plan;
retrieving a plurality of clinical rules from the client's prescription benefit plan;
comparing physical criteria contained in the clinical rule with patient criteria; and
comparing treatment types contained in the clinical rules with a treatment type specified in the prescription claim.

40. A method of evaluating prescription claims using predefined clinical rules, comprising the steps:
determining if one or more prescription products in a prescription claim are included in a client's prescription benefits plan;
retrieving a plurality of clinical rules from the client's prescription benefit plan;
comparing physical criteria contained in the clinical rule with patient criteria;
comparing treatment types contained in the clinical rules with a treatment type specified in the prescription claim;
comparing prescription products in the patients clinical history with the prescription products in the prescription claim; and
identifying adverse reactions that can result from using the prescription products in the patient's clinical history simultaneously with the prescription products in the prescription claim.

41. A system for creating clinical rules, comprising:
a computer system;
at least one additional computer; and
a communication interface coupled to said computer system for establishing a communication channel with said at least one additional computer;
said computer being configured to facilitate:
selection of at least one service classification for a clinical rule,
selection of at least one prescription product for the clinical rule,
selection of at least one treatment type for each selected prescription product,
selection of predetermined medical conditions treatable by the prescription products under each clinical rule, based on the defined service classifications, and
specification of at least one coverage criteria for approving coverage of each selected prescription product.

42. A system for creating clinical rules, comprising:
a computer system;
at least one additional computer system; and
a communication interface coupled to said computer system for establishing a communication channel with said at least one additional computer system;
said computer system being configured to:
receive information corresponding to a prescription claim from said at least one additional computer;
determine if one or more prescription products in said prescription claim are included in a client's prescription benefits plan;
retrieve a plurality of clinical rules from the client's prescription benefit plan; and
apply an appropriate clinical rule to each prescription product to determine if said prescription claim should be covered, at least in part, with respect to said prescription products.

43. A method of creating clinical rules for approving coverage of prescription products comprising at least one of the sequential, non-sequential, and sequence independent steps:
selecting at least one service classification for at least one clinical rule;
selecting at least one prescription product for the at least one clinical rule;
selecting at least one treatment type for the at least one prescription product selected;
selecting predetermined medical conditions treatable by the at least one prescription product for the at least one clinical rule, based on the at least one service classification selected; and
specifying at least one coverage criteria for approving coverage of the at least one prescription product selected.

44. A method of evaluating prescription claims using predefined clinical rules, comprising the steps:
determining whether at least one prescription product in a prescription claim are included in a prescription benefits plan of at least one client;
retrieving a plurality of clinical rules from the prescription benefit plan;
determining a treatment type for each prescription product in the prescription claim; and
applying at least one of the plurality of clinical rules to the at least one prescription product to determine whether the prescription claim is reimbursable, at least in part, with respect to the at least one prescription product.
